(19) United States
(12) Patent Application Publication
(10) Pub. No.: US 2010/0324451 A1
(43) Pub. Date: Dec. 23, 2010

(54) BLOOD TESTING APPARATUS

(75) Inventors: Hiroko Ishibashi, Ehime (JP); Toshiki Matsumoto, Ehime (JP); Kenichi Hamanaka, Ehime (JP); Keisuke Matsumura, Ehime (JP); Tatsuhiko Furukawa, Ehime (JP)

Correspondence Address: GREENBLUM & BERNSTEIN, P.L.C., 1950 ROLAND CLARKE PLACE, RESTON, VA, 20191, US

(73) Assignee: PANASONIC CORPORATION, Osaka (JP)

(21) Appl. No.: 12/526,343

(22) PCT Filed: Feb. 8, 2008
(86) PCT No.: PCT/JP2008/000187

§ 371 Date: Aug. 7, 2009

(30) Foreign Application Priority Data
Feb. 9, 2007 (JP) 2007-030017

(51) Int. Cl. A61B 5/151

(52) U.S. Cl. 600/583

(57) ABSTRACT

Provided is a blood testing apparatus capable of measuring a blood sugar level while keeping the operability, even if the remaining battery life is short. This blood testing apparatus includes a casing (12) having a cylindrical opening cylinder (12b), a blood sensor (22) mounted removably in the opening cylinder (12b) of the casing (12) for analyzing the blood, a blood introducing portion (34) formed in the blood sensor (22) and having an opening for storing this opening with the blood having flown from the skin by a pierce, a laser emitting device (13) disposed in the casing (12) for causing a laser beam to pierce the skin through the inside of the opening cylinder (12b) of the casing and the opening of the blood introducing portion (34), and a needle piercing unit (18) disposed in the casing for piercing the skin with the needle through the opening of the blood introducing portion (34).

### TECHNICAL FIELD

The present invention relates to a blood test apparatus for testing the property of blood and the so on.

### BACKGROUND ART

Diabetes patients need to measure their blood sugar level on a regular basis and inject insulin based on the measured blood sugar level to maintain a normal blood sugar level. To maintain this normal blood sugar level, diabetes patients need to measure the blood sugar level on a regular basis, and sample a small amount of blood from their fingertips using a blood test apparatus and measure the blood sugar level from the sampled blood

A shown in FIG. 1, a conventional blood test apparatus has: housing 2; cylindrical body 2a forming this cylindrical body 2; puncturing opening part 2c provided at the front end of this cylindrical body 2a; laser emitting apparatus 3 provided inside cylindrical body 2; blood sensor 4 (hereinafter, referred to as “sensor”) that is provided facing this laser emitting apparatus 3 and that has blood guiding part 4a; electrical circuit section 5 connected to this blood sensor 4; and electric battery 6 that supplies power to this electrical circuit section 5 and laser emitting apparatus 3.

The operation of blood test apparatus 1 constituted as described above will be explained below. As shown in FIG. 2, for example, blood test apparatus 1 is held by the right hand and is abutted on skin 7 of the left hand. Then, puncturing button 3b shown in FIG. 1 is pressed. Then, laser light 3a is emitted from laser emitting apparatus 3. This laser light 3a punctures skin 7. As a result of this puncturing, blood 8 flows out from skin 7. This blood 8 is temporarily stored in blood guiding part 4a provided in sensor 4. The blood sugar level of blood 8 stored in this blood guiding part 4a is measured in electrical circuit section 5 provided inside blood test apparatus 1.

In this way, conventional blood test apparatus 1 uses laser emitting apparatus 3 as a puncturing means and, consequently, there is no burden of replacing the puncturing needle every puncturing. Further, each time electric battery 6 is consumed and its remaining power is decreased, electric battery 6 needs to be replaced with new electric battery (not shown).

Furthermore, for example, Patent Literature 1 is known as prior art reference information related to the present invention.

### Citation List

### Patent Literature

PTL 1: Japanese Patent Application Laid-Open No. 2004-533866

### SUMMARY OF INVENTION

### Technical Problem

However, such conventional blood test apparatus 1 uses laser emitting apparatus 3 and does not need to replace a puncturing needle, but consumes great power. Further, when electric battery 6 is consumed and its remaining power is decreased, puncturing by the laser emitting apparatus is not possible. As a result, the blood sugar level cannot be measured and administering an adequate dose of insulin becomes difficult. Therefore, the disease may be likely to worsen.

Further, a puncturing method using a simple needle to be used upon emergency has poor operability and unreliability.

The present invention solves such a problem, and, to measure the blood sugar level without deteriorating the operability even when remaining power of the electric battery is decreased, the object of the present invention is to provide a blood test apparatus formed such that an emergency puncturing means can be mounted.

### Solution to Problem

To achieve this object, the blood test apparatus according to the present invention employs a configuration which includes: a housing that has an open body of a cylindrical shape of a cylindrical shape; a blood sensor that can be attached detachably to the open body of a cylindrical shape of the housing and that analyzes blood; a blood guiding part that is formed in the blood sensor, that has an opening part and that stores blood which flows out from skin as a result of puncturing, in the opening part; a laser emitting apparatus that is provided in the housing and that punctures skin by means of laser light which passes an interior of the open body of a cylindrical shape of the housing and the opening part of the blood guiding part and punctures skin; and a needle-puncturing apparatus that is provided in the housing and that punctures skin by means of a needle which passes the opening part of the blood guiding part and punctures skin.

### Advantageous Effects of Invention

The present invention can selectively use for a puncturing means a laser emitting apparatus that requires a supply of power or a needle-puncturing apparatus that does not require a supply of power, so that it is possible to measure the blood sugar level without deteriorating the operability even when remaining power of the electric battery is decreased.

Moreover, the laser emitting apparatus and needle-puncturing apparatus both can puncture skin by means of laser light and a puncturing needle that pass the vicinity of the blood guiding part, and, consequently, perform blood test using the same blood sensor. Accordingly, for example, a blood sensor needs not be prepared separately, and the same blood sensor can be used in both the laser emitting apparatus and needle-puncturing apparatus. That is, even when the puncturing means changes, another blood sensor needs not to be prepared, so that the burden on a user decreases.

### BRIEF DESCRIPTION OF DRAWINGS

FIG. 1 is a cross-sectional view of a conventional blood test apparatus;

FIG. 2 illustrates the state of use of the conventional blood test apparatus;

FIG. 3 is a cross-sectional view of a blood test apparatus according to Embodiment 1 of the present invention;

FIG. 4 is a cross-sectional view of a puncturing unit constituting the blood test apparatus according to Embodiment 1 of the present invention;

FIG. 5 is a cross-sectional view of an A-A line of the puncturing unit according to Embodiment 1 of the present invention;

FIG. 6 is a perspective view of the puncturing unit according to Embodiment 1 of the present invention;

FIG. 7 is a cross-sectional view and perspective plan view of a sensor unit according to Embodiment 1 of the present invention;

FIG. 8 is a plan view of a sensor unit seen from the bottom surface according to Embodiment 1 of the present invention;

FIG. 9 is an exploded plan view of guiding parts according to Embodiment 1 of the present invention; FIG. 10 is a cross-sectional view of a sensor according to

Embodiment 1 of the present invention;

FIG. 11 is a perspective plan view of the sensor according to Embodiment 1 of the present invention (in case where the sensor is hexagonal);

FIG. 12 is a plan view of components constituting the sensor according to Embodiment 1 of the present invention;

FIG. 13 is a cross-sectional view of main parts in the sensor according to Embodiment 1 of the present invention;

FIG. 14 is a plan view of main parts in the sensor according to Embodiment 1 of the present invention;

FIG. 15 is a cross-sectional view of the sensor according to Embodiment 1 of the present invention in the first state;

FIG. 16 is a cross-sectional view of the sensor according to Embodiment 1 of the present invention in the second state;

FIG. 17 is a cross-sectional view of the sensor according to Embodiment 1 of the present invention in the third state;

FIG. 18 is a cross-sectional view and perspective plan view of another sensor unit according to Embodiment 1 of the present invention;

FIG. 19 is a cross-sectional view and perspective plan view of another example of the sensor unit according to Embodiment 1 of the present invention;

FIG. 20 is a cross-sectional view and perspective plan view of another example of the sensor unit according to Embodiment 1 of the present invention;

FIG. 21 is a cross-sectional view showing the entire blood test apparatus, to which the puncturing unit mounting the sensor unit shown in FIG. 7, FIG. 18 and FIG. 19 is attached, according to Embodiment 1 of the present invention;

FIG. 22 is a cross-sectional view showing the entire blood test apparatus, to which the puncturing unit mounting the sensor unit shown in FIG. 20 is attached, according to Embodiment 1 of the present invention;

FIG. 23 is a cross-sectional view of the laser emitting apparatus constituting the blood test apparatus according to Embodiment 1 of the present invention;

FIG. 24 is a block diagram showing an electrical circuit section and its vicinity according to Embodiment 1 of the present invention;

FIG. 25 illustrates the operation according to Embodiment 1 of the present invention;

FIG. 26 is a cross-sectional view of the blood test apparatus according to Embodiment 2 of the present invention;

FIG. 27 is a cross-sectional view of the blood test apparatus according to Embodiment 3 of the present invention;

FIG. 28 is a cross-sectional view of the blood test apparatus according to Embodiment 4 of the present invention; and

FIG. 29 is a cross-sectional view of the blood test apparatus according to Embodiment 5 of the present invention.

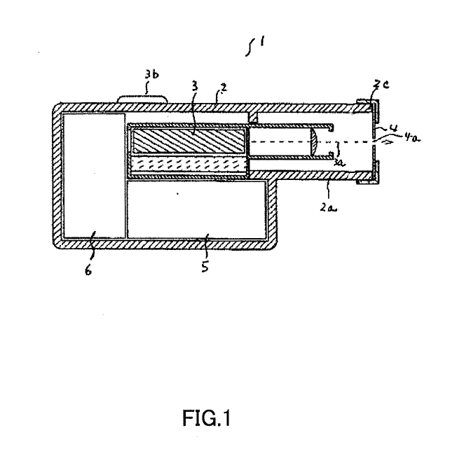

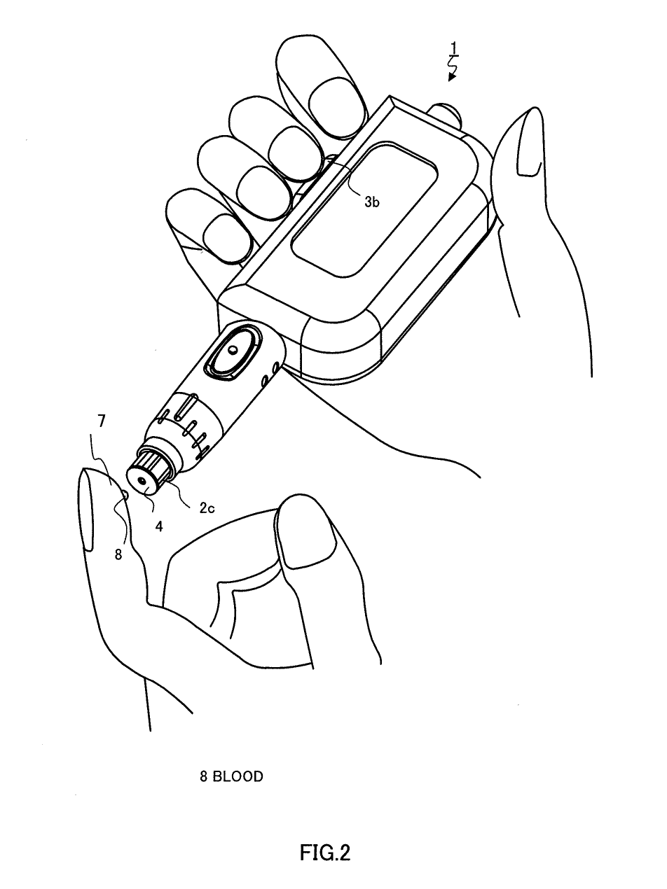

### DESCRIPTION OF EMBODIMENTS

Hereinafter, embodiments of the present invention will be explained based on the accompanying drawings.

### Embodiment 1

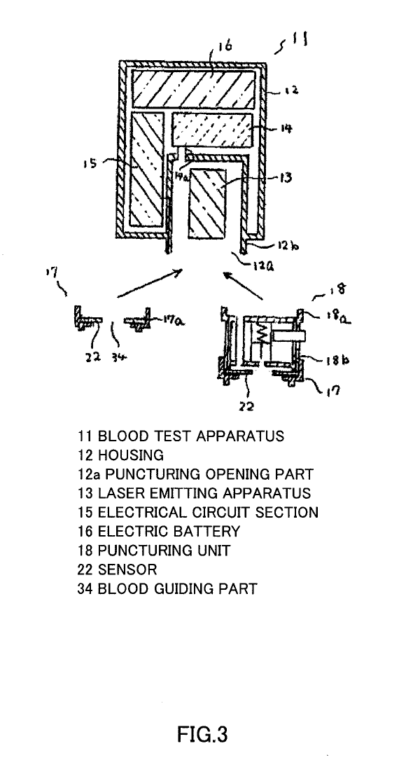

FIG. 3 is a cross-sectional view showing blood test apparatus 11 according to Embodiment 1 of the present invention. In FIG. 3, housing 12 is made of a resinic material and is provided with cylindrical body 12b of a cylindrical shape that has puncturing opening part 12a. Laser emitting apparatus 13 is attached inside this cylindrical body 12b. Further, vacuuming means 14 continuing to vacuuming passage 14a, is attached to cylindrical body 12b. Further, electrical circuit section 15 is provided next to cylindrical body 12b. Electric battery 16 is replacably accommodated at one end of housing 12 at the other end of which puncturing opening part 12a is located.

Sensor unit 17 is attached detachably to puncturing opening part 12a. This sensor unit 17 is constituted by holder 17a and blood sensor 22 (hereinafter “sensor”), and sensor 22 is attached inside this holder 17a detachably. In virtually the center of sensor 22, blood guiding part 34 that stores blood 8 is formed.

Puncturing unit 18 is a unit that punctures skin by means of a needle (an example of a needle-puncturing apparatus is used here), and is attached detachably between puncturing opening part 12a and sensor unit 17 in blood test apparatus 11. That is, upper part 18a of puncturing unit 18 is attached detachably to puncturing opening part 12a, and sensor unit 17 is attached detachably to lower part 18b of puncturing unit 18. That is, both sensor unit 17 and puncturing unit 18 can be attached to puncturing opening part 12a. When a needle-puncturing unit is attached, use of laser emitting apparatus 13, which is built in the blood test apparatus, is automatically prevented. That is, power supply to laser emitting apparatus 13 from electric battery 16 and control signals to laser emitting apparatus 13 are automatically stopped or blocked by electrical circuit section 15.

Consequently, it is possible to selectively perform puncturing by attaching sensor unit 17 directly to puncturing opening part 12a and using laser emitting apparatus 13, and puncturing by attaching puncturing unit 18 to puncturing opening part 12a and sensor unit 17 to puncturing unit 18 and using a needle-puncturing apparatus. That is, in case where puncturing unit 18 is used, puncturing is performed by means of a puncturing needle, so that it is possible to perform puncturing without using electric battery 16. Consequently, even when electric battery 16 is consumed and its remaining power is decreased, it is still possible to measure the blood sugar level. Accordingly, it is possible to adequately prevent the disease from worsening.

Further, laser emitting apparatus 13 and puncturing unit 18 both make laser light and a puncturing needle pass near blood guiding part 34 to perform puncturing, so that it is possible to test blood 8 using same sensor 22. Accordingly, for example, another sensor needs not to be prepared and, even when the puncturing means changes, the burden on the user decreases. Further, the vicinity of blood guiding part 34 generally refers to the range of 0.5 millimeters to 5 millimeters around blood guiding part 34.

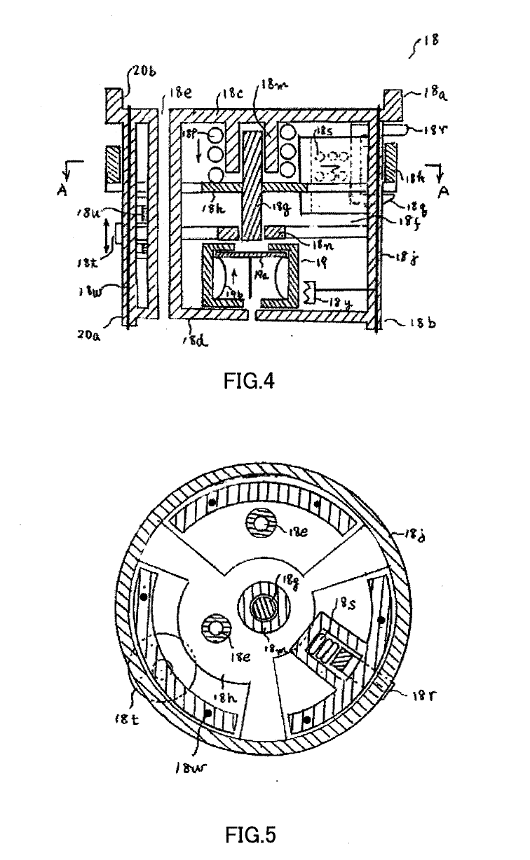

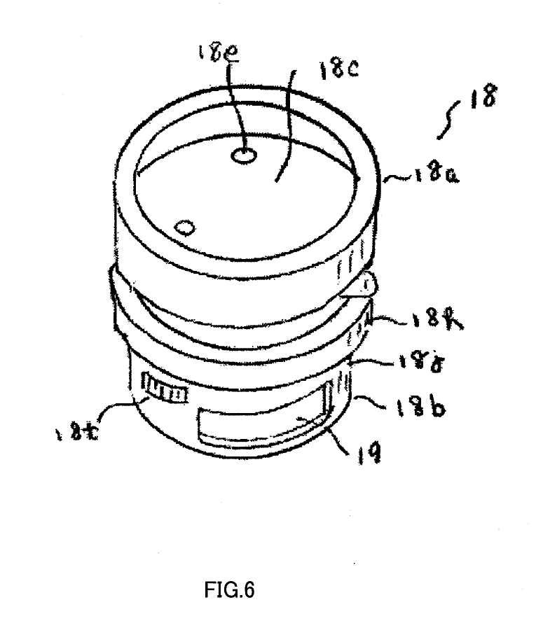

Next, puncturing unit 18 will be explained in detail using FIGS. 4, 5 and 6. FIG. 4 is a cross-sectional view showing puncturing unit 18 from the side, and FIG. 5 is a cross-sectional view cutting puncturing unit 18 in the A-A plane and showing puncturing unit 18 from above. Further, FIG. 6 is a perspective view of puncturing unit 18. Puncturing unit 18 has a cylindrical shape and is sealed by upper surface 18c and lower surface 18d. Further, these upper surface 18c and lower surface 18d communicate through two vacuuming passages 18e. Consequently, even when middle part 18f between upper surface 18c and lower surface 18d is opened to air, it is possible to apply a negative pressure from upper surface 18c to lower surface 18d.

Guard 18h formed in stick 18g is led outside cylinder 18j to form handle 18k. Guide 18m is formed integrally with upper surface 18c, and guides stick 18g so as to slide in the up and down direction. Further, guide 18n is formed to extend from cylinder 18j, and guides stick 18g so as to slide only in the up and down direction in conjunction with guide 18m.

Springs 18p are inserted between upper surface 18c and guard 18h to urge stick 18g downward. Latch claw 18q of handle 18k is formed integrally with puncturing button 18r. These latch claw 18q and puncturing button 18r are urged by springs 18s toward the outside of cylinder 18j.

Puncturing depth adjusting knob 18t provided in cylinder 18j moves guard 18h on screws 18u to define the location where guard 18 stops. By defining the location where guard 18h stops, the distance stick 18g falls is defined and the depth to which the puncturing needle punctures skin is adjusted.

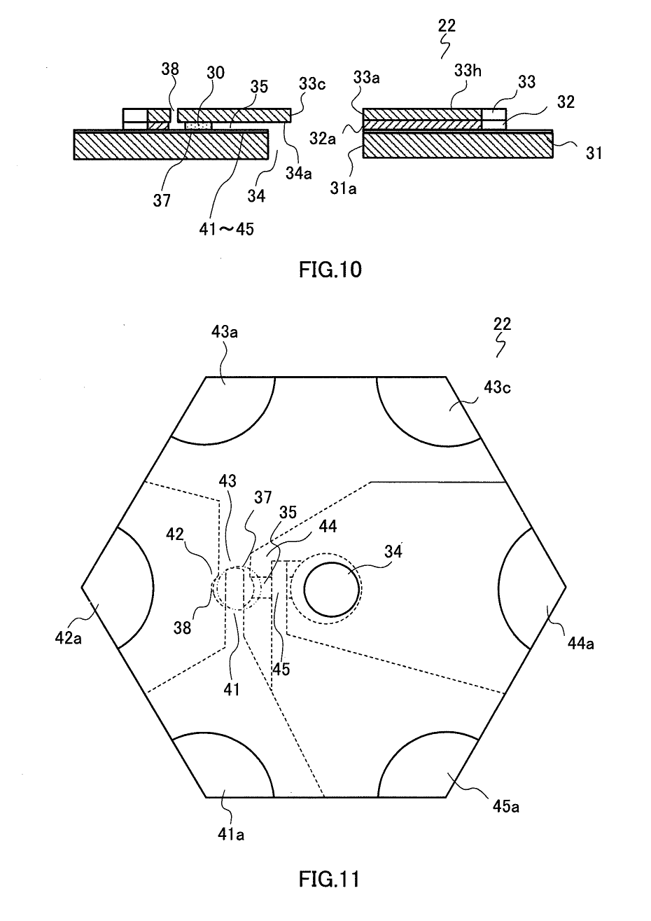

A plurality of conductors 18w, which communicate upper part 18a with lower part 18b, lead signals from sensor 22 to electrical circuit section 15 through puncturing opening part 12a. These conductors 18w are connected to connector electrodes in upper part 18a and lower part 18b, and these connector electrodes are provided evenly in a circle. Further, these connector electrodes contact and connect with connectors 53 (53a to 53g) formed in puncturing opening part 12a in upper part 18a, and contact and connect with connection electrodes 41a to 45a (see FIG. 11) formed in sensor 22 in lower part 18b.

Further, in the inner surface of the ring formed in upper part 18a and in the outer surface of lower part 18b, guide parts 20 (see FIG. 9) are formed to define the rotation angle when the puncturing unit is inserted, and make the connector electrodes abut on upper part 18a and lower part 18b reliably. Needle unit 19 is inserted removably between stick 18g and lower surface 18d. Consequently, it is possible to readily replace needle unit 19. Inside this needle unit 19, puncturing needle 19a is urged upward by springs 19b. Detecting sensor 18y detects whether or not there is needle unit 19. Signals outputted from this detecting sensor 18y are connected to the connector electrodes through conductors 18w.

Next, the operation of this puncturing unit 18 will be explained. First, handle 18k slides upward against springs 18p. Then, latch claw 18q latches with handle 18k. In this state, needle unit 19 is inserted. Then, puncturing button 18r is pressed. Then, latch claw 18q and handle 18k are disengaged, so that stick 18g hits puncturing needle 19a of puncturing unit 19. Puncturing needle 19a passes blood guiding part 34 of sensor 22 and punctures skin 7. Further, the puncturing depth is adjusted in advance by knob 18t.

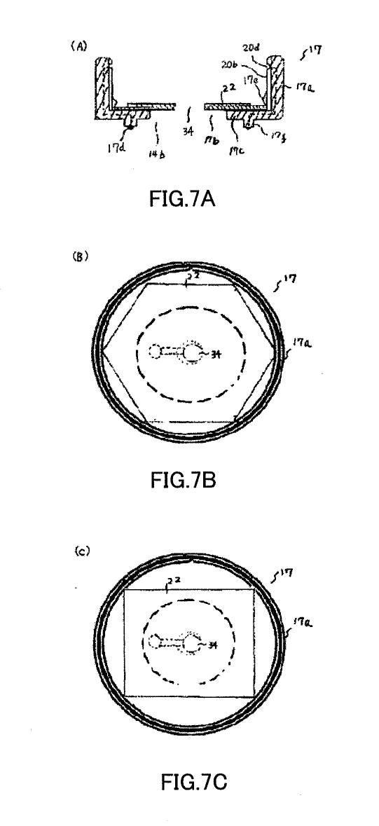

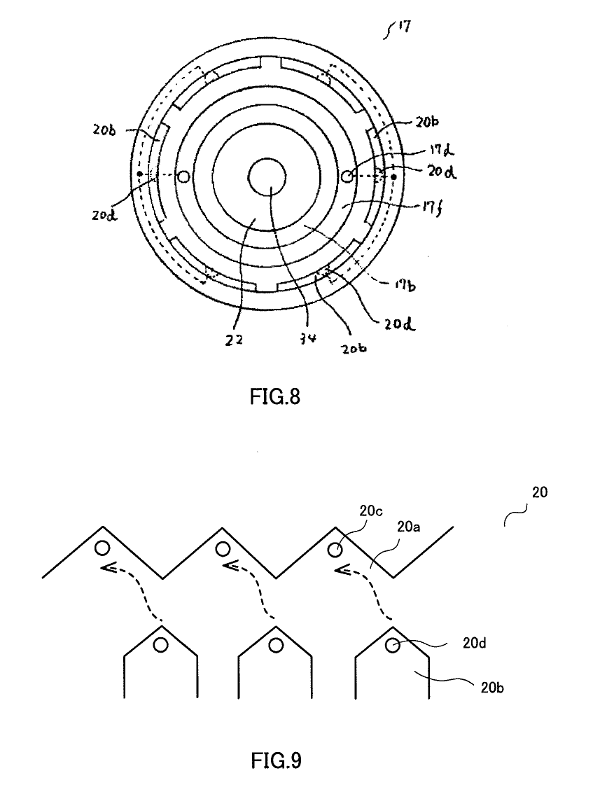

FIG. 7A is a cross-sectional view of sensor unit 17 of one example, and FIG. 8 is a plan view showing FIG. 7(A) from the bottom surface. Sensor unit 17 is constituted by holder 17a and sensor 22 that is inserted in this holder 17a. Inside holder 17a, receiving board 17c having hole 17b in the center is provided and sensor 22 is mounted on this receiving board 17c. Further, this sensor 22 is latched by latching convex parts 17e formed inside holder 17a. Further, convex part 17f of a ring shape is formed below receiving board 17c, forming vacuuming chamber 14b.

Convex part 17f is provided with skin detecting sensors 17d that detect contact with skin. Signals from these skin detecting sensors 17d are connected with concave parts 20d formed in guides 20b through conductor wires. Skin detecting sensors 17d detect the resistance when skin detecting sensors 17d abut on skin 7, and are constituted by conductor electrodes. Then, as shown in FIG. 8, skin detecting sensors 17d are connected such that a plurality of concave parts 20d are divided into two. This is to extract signals irrespective of the insertion direction of sensor unit 17 by acquiring signals from convex parts 20c that are located 180 degrees apart and that fit in these concave parts 20d. Further, this relationship is possible even when concave parts 20d formed in sensor unit 17 and convex parts 20c formed in puncturing opening part 12a are switched.

FIG. 7(B) and FIG. 7(C) are perspective plan views showing sensor unit 17 shown in FIG. 7(A) from above. FIG. 7(B) shows a case where the shape of sensor 22 is hexagonal and blood guiding part 34 is in virtually the center of sensor 22. FIG. 7(C) shows a case where the shape of sensor 22 is a square and blood guiding part 34 is provided in virtually the center of sensor 22.

FIG. 9 is an exploded plan view of guiding parts 20. These guiding parts 20 are formed between puncturing opening part 12a and sensor unit 17, between puncturing opening part 12a and upper part 18a of puncturing unit 18 and between lower part 18b of puncturing unit 18 and sensor unit 17, in order to make electrodes contact each other to lead signals from sensor unit 22 even when sensor unit 17 or puncturing unit 18 and sensor unit 17 both are attached to puncturing opening part 12a carelessly.

As shown in FIG. 9, guides 20a of a concave shape are provided in the outer surface of cylindrical body 12b forming puncturing opening part 12a and in the outer surface of lower part 18b of puncturing unit 18. Further, guides 20b of a convex shape are provided in the inner surface of sensor unit 17 and in the inner surface of upper part 18a of puncturing unit 18. Consequently, even when sensor unit 17 or puncturing unit 18 is inserted carelessly, the direction of sensor unit 17 or puncturing unit 18 is corrected along these guides 20a and 20b. Consequently, the electrodes are reliably connected with each other, so that it is possible to lead signals from sensor 22 to electrical circuit section 15.

Convex parts 20c are formed in the depth parts of guides 20a, and are electrically conductive and elastic. Concave parts 20d are provided at the front ends of guides 20b, and are electrically conductive. These convex parts 20c and concave parts 20d fit, thereby positioning sensor unit 17 and puncturing unit 18 and leading signals from skin detecting sensors 17d attached to sensor unit 17 through these convex parts 20c and concave parts 20d, to electrical circuit section 15.

FIG. 10 is a cross-sectional view of sensor 22 attached to sensor unit 17. This sensor 22 is constituted by substrate 31, spacer 32 pasted on the upper surface of this substrate 31 and cover 33 pasted on the upper surface of spacer 32, and has a plate shape.

Substrate hole 31a formed in virtually the center of substrate 31, spacer hole 32a formed in virtually the center of spacer 32 and cover hole 33a formed in virtually the center of cover 33 communicate to form blood guiding part 34 of blood 8. This blood guiding part 34 is open downward to abut on skin 7 and sample blood 8. One end of supply channel 35 for blood 8 continues to this blood guiding part 34 and supply channel 35 leads blood 8 stored in blood guiding part 34 by capillary action to detecting section 37 formed on supply channel 35 (see FIG. 11). Further, the other end of this supply channel 35 continues to air hole 38.

Here, a water-repellant material is used for upper surface 33h of cover 33. Further, a hydrophillic material is used inside supply channel 35. Here, preferably, ceiling 34a of blood guiding part 34 is treated to be less hydrophilic than supply channel 35 or treated to be less water-repellant than upper surface 33h of cover 33.

Reagent 30 is arranged on detecting section 37. This reagent 30 can be obtained by adding and dissolving PQQ-GDH (0.1 to 5.0 U/sensor), potassium ferricyanide (10 to 200 millimole), maltitol (1 to 50 millimole) and taurine (20 to 200 millimole) in a CMC solution of 0.01 to 2.0 wt % to prepare a reagent solution and by dropping the reagent solution and drying reagent 30 on the detecting section.

FIG. 11 is a perspective plan view of sensor 22. The shape of sensor 22 is a regular hexagon, and connection electrodes 41a to 45a that are connected with connectors 53a to 53f provided in puncturing opening part 12a of blood test apparatus 11, and reference electrode 43c that is connected with connection electrode 43a, are formed in the respective six apexes of this regular hexagon.

In blood guiding part 34, supply channel 35, one end of which is connected with this blood guiding part 34, is provided toward detection electrode 42. Further, the other end of this supply channel 35 continues to air hole 38. On this supply channel 35, there are, from the side closer to blood guiding part 34, detection electrode 44 connected with connection electrode 44a, detection electrode 45 connected with connection electrode 45a, detection electrode 44, which is provided again, connected with connection electrode 44a, detection electrode 43 connected with connection electrode 43a and reference electrode 43c, detection electrode 41 connected with connection electrode 41a, detection electrode 43, which is provided again, connected with connection electrode 43a and reference electrode 43c and detection electrode 42 connected with connection electrode 42a. Further, reagent 30 (see FIG. 10) is arranged on detection electrodes 41 and 43.

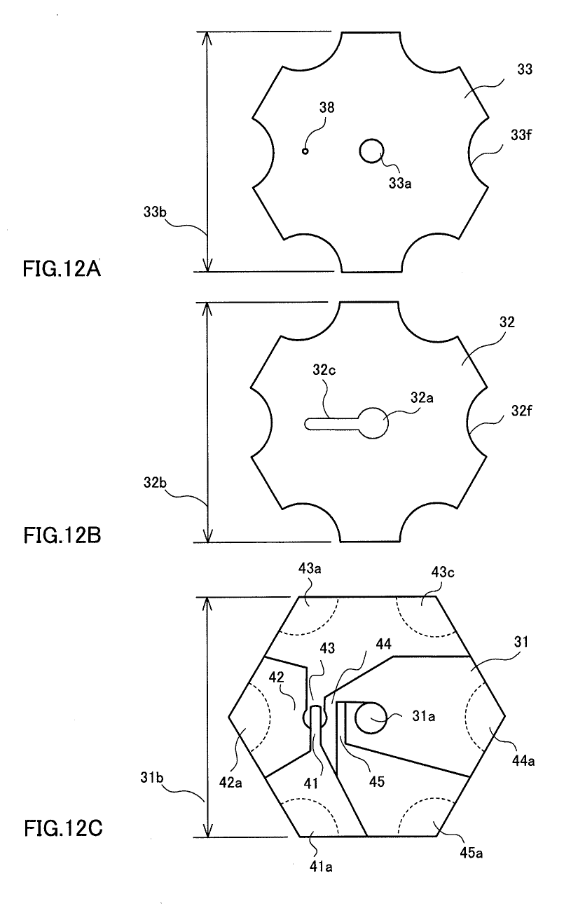

FIG. 12 is an exploded plan view of sensor 22. FIG. 12(C) is a plan view of regular hexagonal substrate 31 constituting sensor 22, and its dimension 31b is about 9 millimeters. The material of this substrate 31 is polyethylene terephthalate (PET) and the thickness of substrate 31 is about 0.100 millimeters.

An electrically conductive layer is formed on the upper surface of this substrate 31 by the sputtering method or the vapor deposition method using material such as gold, platinum, or palladium, and detection electrodes 41 to 45 and connection electrodes 41a to 45a and reference electrode 43c derived from these detection electrodes 41 to 45 are integrally formed by applying laser machining to this electrically conductive layer. Substrate hole 31a is provided in virtually the center of substrate 31.

FIG. 12(B) is a plan view of spacer 32 and its dimension 32b is about 9 millimeters. Spacer hole 32a is provided in virtually the center of spacer 32 in a position to meet substrate hole 31a. This spacer 32 is formed by machining a regular hexagon, and six semicircular notches 32f are formed in the six apexes of this regular hexagon to meet connection electrodes 41a to 45a and reference electrode 43c of substrate 31.

Further, slit 32c is formed to continue to this spacer hole 32a and this slit 32c forms supply channel 35 for blood 8. The wall surfaces of this slit 32c and the upper surface of substrate 31 to meet the wall surfaces of slit 32c are subjected to hydrophilic treatment. The width of this slit 32c is made about 0.600 millimeters and the length of slit 32c is made about 2.400 millimeters to form supply channel 35 with a cavity of about 0.144 microliters. In this way, it is possible to perform test with a small amount of blood 8, so that patients do not have to get strained and scared. The material of spacer 32 is polyethylene terephthalate and the thickness of spacer 32 is about 0.050 millimeters.

FIG. 12(A) is a plan view of cover 33. Its dimension 33b is about 9 millimeters. Cover hole 33a is provided in a position slightly decentered from the center of cover 33. Air hole 38 is provided to meet the front end part of supply channel 35. Diameter 38a of this air hole 38 is about 50 micrometers. The reason for reducing the diameter of air hole 38 in this way is to prevent blood 8 from flowing out from air hole 38. Cover 33 is formed by machining a regular hexagon, and six semicircular notches 33f are formed in the six apexes of this regular hexagon, which is not machined yet, to meet connection electrodes 41a to 45a and reference electrode 43c of substrate 31. The material of this cover 33 is polyethylene terephthalate and the thickness of cover 33 is about 0.075 millimeters.

Substrate 31, spacer 32 and cover 33 constituting sensor 22 can each be formed by dividing a parent substrate of a fixed measure into several pieces. These substrate 31, spacer 32 and cover 33 that are divided are regular hexagons and, consequently, can be aligned in the parent substrates without space. Accordingly, these materials are each efficiently scribed in the parent substrate, which cuts waste, is economical and contributes to resource saving.

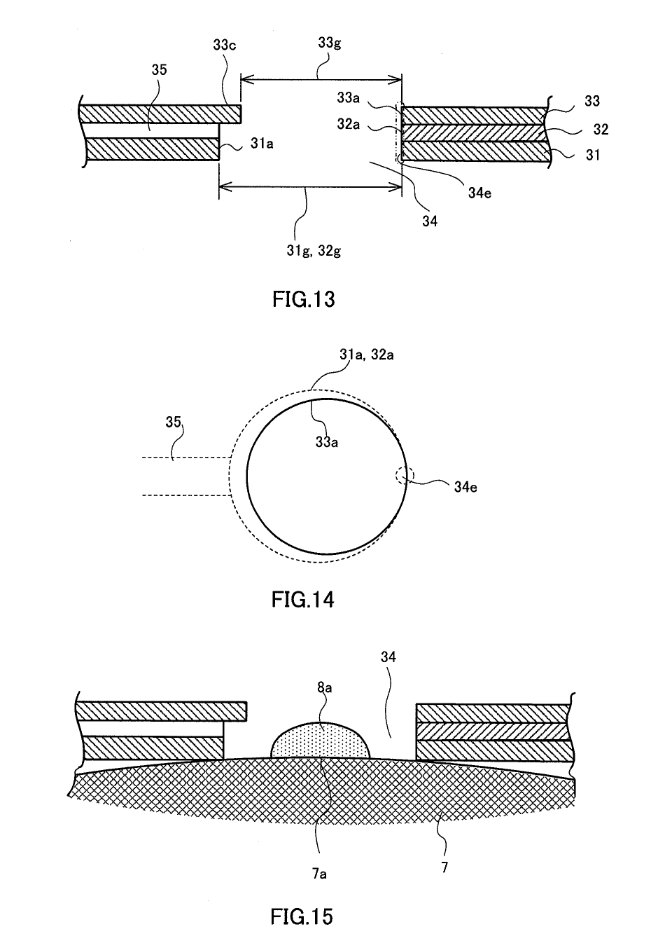

FIG. 13 is a cross-sectional view in the vicinity of blood guiding part 34 of sensor 22 and FIG. 14 is a plan view of blood guiding part 34. In FIG. 13 and FIG. 14, diameter 31g of substrate hole 31a formed in substrate 31 and diameter 32g of spacer hole 32a formed in spacer 32 are about 1.750 millimeters, and diameter 33g of cover hole 33a formed in cover 33 is 1.500 millimeters. The centers of substrate hole 31a and spacer hole 32a are on the same line, and the center of cover hole 33a is in a direction slightly apart from the supply channel 35 side. Further, opposite side 34e of supply channel 35 in substrate hole 31a, spacer hole 32a and cover hole 33a are on the same plane.

According to this configuration, projecting part 33c projecting from supply channel 35 toward the center of blood guiding part 34 is formed in blood guiding part 34. The dimensions of projection of this projecting part 33c is 0.250 millimeters and is 0.100 millimeters greater than the sum, 0.150 millimeters, of the thicknesses of substrate 31 and spacer 32.

Further, opposite side 34e of supply channel 35 in blood guiding part 34 is formed on the same plane. That is, there are the centers of substrate hole 31a and spacer hole 32a in the center of blood guiding part 34 and the center of cover hole 33a on the opposite side of supply channel 35. The relationship between diameters 31g, 32g and 33g of these holes are that diameter 31g of substrate hole 31a and diameter 32g of spacer hole 32a are equal and diameter 33g of cover hole 33a is smaller than diameter 32g of spacer hole 32a.

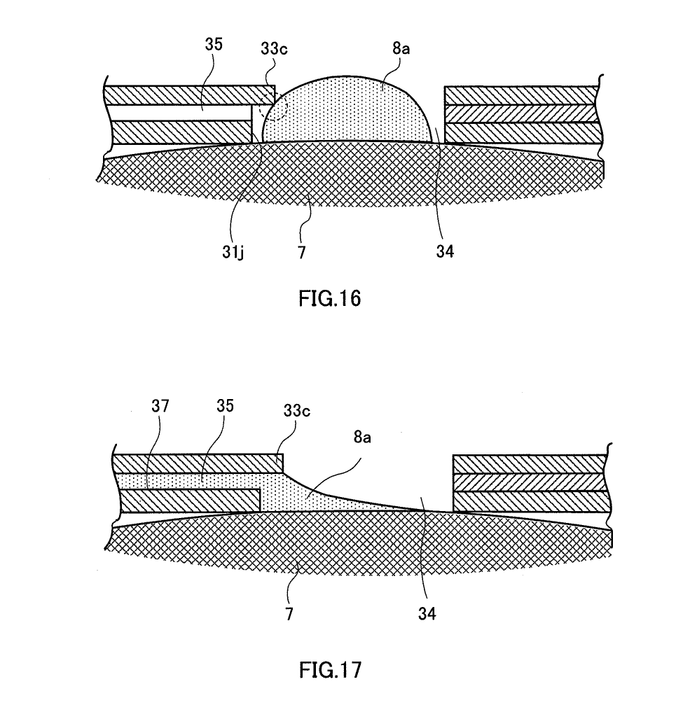

The operation of sensor 22 constituted as described above will be explained below. As shown in FIG. 15, when skin 7 inside blood guiding part 34 is punctured, blood 8 flows out from punctured hole 7a by this puncturing to form blood drop 8a. As shown in FIG. 16, this blood drop 8a increasingly grows, and abuts on the tip of projecting part 33c (shown by the dotted line). Further, before blood drop 8a grows to reach contact point 31j with skin 7 on the supply channel 35 side, as shown in FIG. 17, blood drop 8a flows into detecting section 37 through supply channel 35, at a burst, in a rate-controlled state, thanks to the capillary action produced by projecting part 33c and skin 7.

In this way, the capillary action produced in the space between cover 33 and skin 7 becomes strong on the supply channel side, so that it is possible to allow blood 8 to flow into detecting section 37 through supply channel 35 in a reliable manner, before blood 8 fills blood guiding part 34. Consequently, it is possible to reduce the amount of blood left in blood guiding part 34. That is, the amount of blood 8 to sample decreases accordingly, so that it is possible to alleviate the burden upon patients.

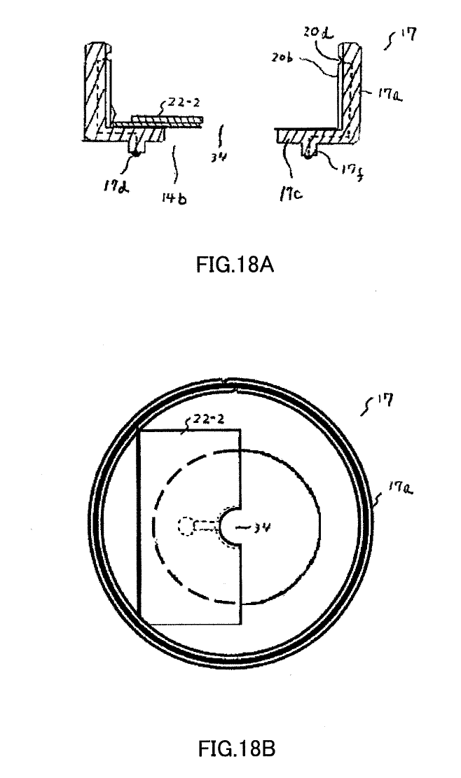

FIG. 18(A) and FIG. 18(B) are a cross-sectional view of sensor unit 17 mounting another example of sensor 22-2 and a perspective plan view showing sensor unit 17 from above.

Sensor 22-2 has a square shape, and blood guiding part 34 is provided in the longitudinal direction of the side surface of the square.

In this way, as shown in FIG. 7(A) to FIG. 7(C), blood guiding part 34 is not necessarily provided in the center part of the sensor and the essential requirement is that blood guiding part 34 is provided in the vicinity of the position to puncture. Further, sensor 22-2 of a square shape shown in FIG. 18(B) may be flexibly arranged at any angle around blood guiding part 34 (i.e. the position to puncture).

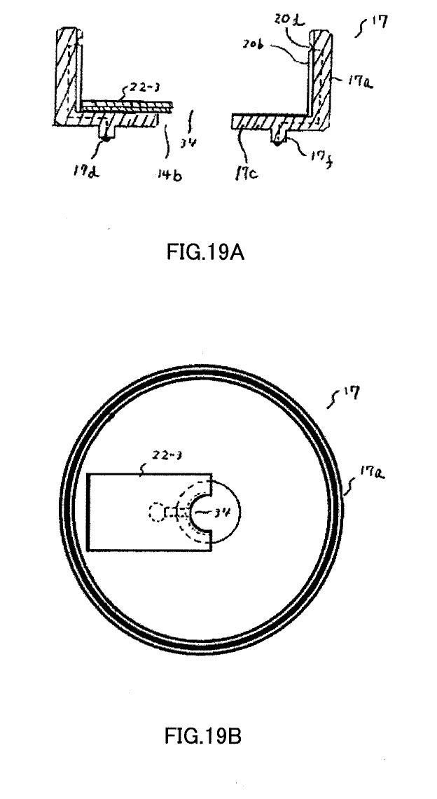

FIG. 19(A) and FIG. 19(B) are a cross-sectional view of sensor unit 17 mounting another example of sensor 22-3 and a perspective plan view showing sensor unit 17 from above. Sensor 22-3 has a square shape, and is the same as in the case of the sensor unit mounting sensor 22-2 described in FIG. 18(A) and FIG. 18(B) except for blood guiding part 34 that is provided in the lateral direction of the side surface of the square.

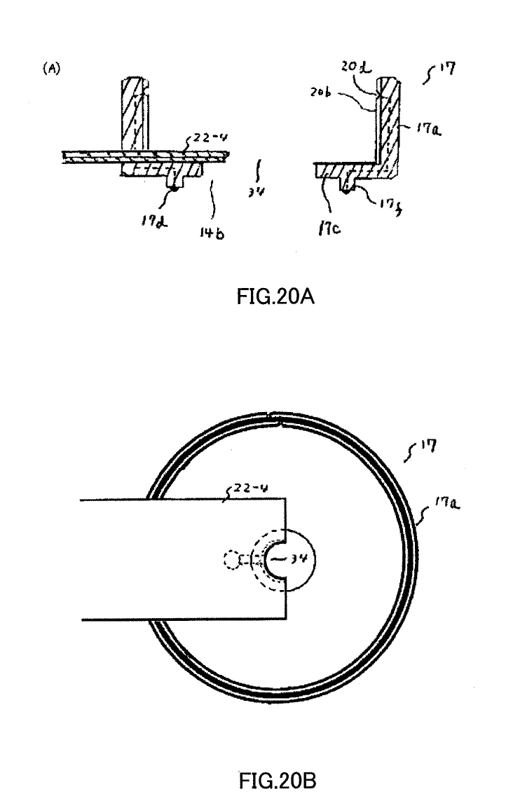

FIG. 20(A) and FIG. 20(B) are a cross-sectional view of sensor unit 17 mounting another example of sensor 22-4 and a perspective plan view showing sensor unit 17 from above.

Although sensor 22-4 has a square shape similar to sensor 22-3 and the location of blood guiding part 34 is the same as in sensor 22-3, the dimensions of sensor 22-4 greater than sensor 22-3 are secured such that sensor 22-4 protrudes from the side surface of sensor unit 17. Compared to the case of sensor 22-3, the operability of attaching sensor 22-4 is good even while the puncturing unit is kept attached. It naturally follows that, in this case, space is provided in the side surface of holder 17a to let in and let out sensor 22-4.

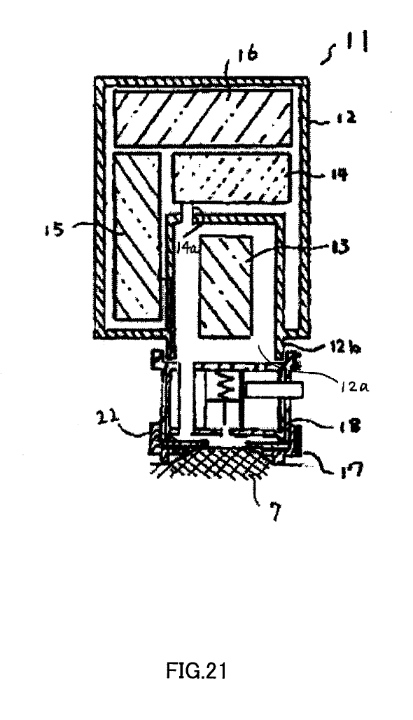

Next, FIG. 21 is a cross-sectional view when the puncturing unit (an example of a needle-puncturing apparatus) mounting sensor unit 17 shown in FIG. 7, FIG. 18 and FIG. 19, is attached to blood test apparatus 11.

In FIG. 21, housing 12 is made of a resinic material and is provided with cylindrical body 12b of a cylindrical shape that has puncturing opening part 12a. Laser emitting apparatus 13 is attached inside this cylindrical body 12b. Further, vacuuming means 14 continuing to vacuuming passage 14a is attached to cylindrical body 12b. Further, electrical circuit section 15 is provided next to cylindrical body 12b. Electric battery 16 is accommodated replacably at one end of housing 12 at the other end of which puncturing opening part 12a is located.

Sensor unit 17 is attached detachably to puncturing opening part 12a. In virtually the center of sensor unit 17, blood guiding part 34 that punctures and guides blood 8 (not shown) is formed.

Further, sensor unit 17 can mount any type of hexagonal or square sensor 22 meeting the blood guiding part in virtually the center of the sensor, square sensor 22-2 meeting the blood guiding part in the longitudinal side of the square sensor and square sensor 22-3 meeting the blood guiding part in the lateral side of the square sensor.

Puncturing unit 18 performs puncturing by means of a puncturing needle and is attached detachably between puncturing opening part 12a and sensor unit 17. That is, upper part 18a of puncturing unit 18 is attached detachably to puncturing opening part 12a of blood test apparatus 11 and sensor unit 17 is attached detachably to lower part 18b of puncturing unit 18.

Consequently, it is possible to selectively perform puncturing by attaching sensor unit 17 directly to puncturing opening part 12a and using laser emitting apparatus 13, and puncturing by attaching puncturing unit 18 to puncturing opening part 12a and sensor unit 17 to puncturing unit 18 and using a needle-puncturing apparatus.

That is, in case where puncturing unit 18 is used, puncturing is performed by means of a puncturing needle, so that it is possible to perform puncturing without using electric battery 16. Consequently, even when electric battery 16 is consumed and its remaining power is decreased, it is possible to measure the blood sugar level. Accordingly, it is possible to adequately prevent the disease from worsening.

Further, laser emitting apparatus 13 and puncturing unit 18 both perform puncturing by means of laser light and a puncturing needle that pass near blood guiding part 34, so that it is possible to test blood 8 using same sensor 22. Accordingly, for example, another sensor needs not to be prepared and, even when a puncturing means changes, the burden on the user decreases. Further, the puncturing unit and the sensor unit have vacuuming passages and can apply negative pressures to the vicinity of the blood guiding part prior to puncturing.

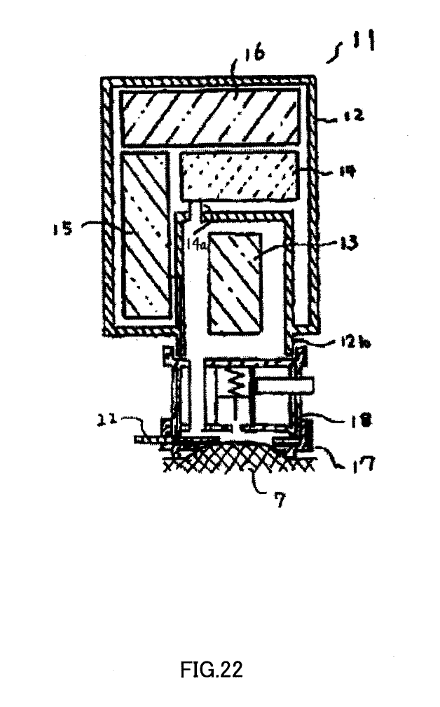

FIG. 22 is a cross-sectional view in the case where the puncturing unit mounting sensor unit 17 shown in FIG. 20 is attached in blood test apparatus 11.

FIG. 22 and FIG. 21 are the same except for sensor unit 17. Sensor unit 17 shown in FIG. 22 has sensor 22-4 meeting blood guiding part 34 in the lateral direction of the side surface of the square of sensor 22-4, and has space for sensor 22-4 because sensor 22-4 has a shape protruding from holder 17a.

Further, puncturing is performed in the vicinity of blood guiding part 34 using laser emitting apparatus 13 and puncturing unit 18 built in blood test apparatus 11. That is, the same position can be punctured using one of both methods, so that the operability is maintained and the reliability is also secured.

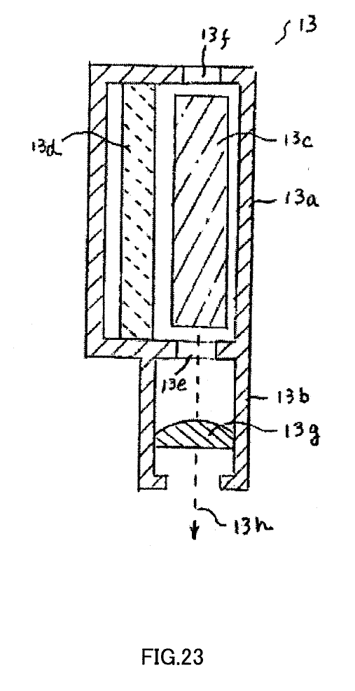

FIG. 23 is a cross-sectional view of laser emitting apparatus 13. Laser emitting apparatus 13 is constituted by oscillating tube 13a and cylindrical body 13b of a cylindrical shape coupled to the front of this oscillating tube 13a. Oscillating tube 13a accommodates Er:YAG (yttrium aluminum garnet) laser crystal 13c and flash light source 13d. Partial transmission mirror 13e of about one percent transmittance is attached to one end of oscillating tube 13a, and total reflection mirror 13f is attached to the other end. Convex lens 13g is attached inside cylindrical body 13b ahead of partial transmittance mirror 13e and is set to adjust the focus of laser light 13h under the skin of the patient.

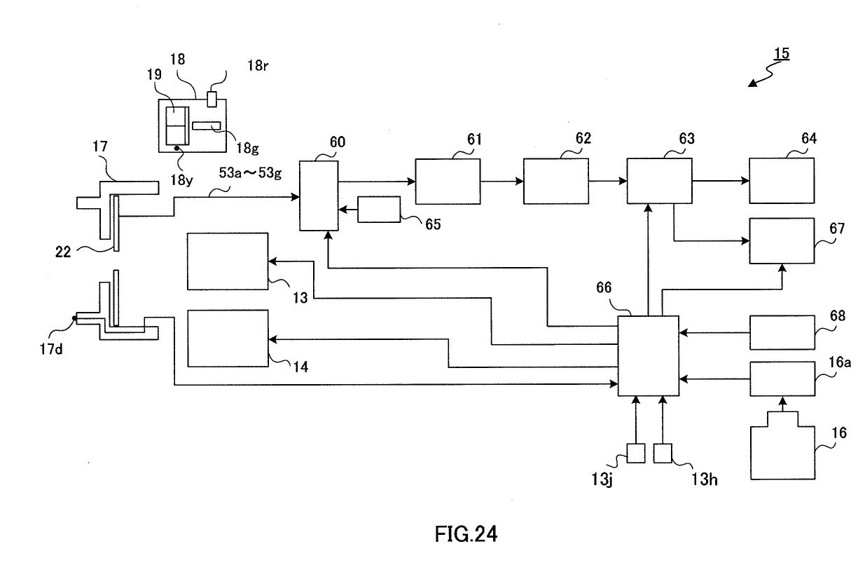

The operation of laser emitting apparatus 13 constituted as described above will be explained below. Puncturing button 13j (see FIG. 24) is pressed. Then, flash light source 13d is excited, and the light source emitted from this flash light source 13d enters Er:YAG laser crystal 13c and is reflected between total reflection mirror 13f, YAG laser crystal 13c and partial transmission mirror 13e to oscillate and amplify. Part of this amplified laser light passes partial transmission mirror 16e by stimulated emission. Laser light 13h that has passed this partial transmission mirror 13e passes lens 13g to pass sensor 22 and adjust its focus inside skin 7. Preferably, the depth of the focus to which laser light punctures skin is between 0.1 millimeters and 1.5 millimeters from skin 7, and is 0.5 millimeters with the present embodiment.

Blood 8 flows out from punctured skin 7. Blood 8 that has flowed out is taken inside sensor 22 and chemically reacts with reagent 30 in this sensor 22. Information about blood 8 that has chemically reacted with reagent 30 is transmitted to electrical circuit section 15 through connectors 53a to 53g and the blood sugar level and the like is calculated in electrical circuit section 15. Further, details of this will be explained later.

With the present embodiment, laser emitting apparatus 13 that enables puncturing without contacting skin 7 of the patient is used as the main puncturing means, so that, in the normal state of use, a puncturing needle needs not to be changed and preparation prior to puncturing becomes simple compared to puncturing apparatuses using a puncturing needle. Further, skin 7 and laser emitting apparatus 13 do not contact, which is sanitary. Furthermore, there are no movable components, and technical malfunction decreases. Moreover, the structure of blood test apparatus 11 can be made water-proof, so that the apparatus can be washed entirely. Further, the puncturing voltage for this laser light 13h is about 300 volts. Accordingly, patients suffer from little pain.

FIG. 24 is a block diagram of electrical circuit section 15. In FIG. 24, connection electrodes 41a to 45a and reference electrode 43c of sensor 22 are connected with switching circuit 60 through connectors 53a to 53g. The output of this switching circuit 60 is connected with the input of current/voltage converter 61. The output of current/voltage converter 61 is connected with the input of calculating section 63 through analogue/digital converter 62 (hereinafter “A/D converter”). The output of this calculating section 63 is connected with display section 64 formed with liquid crystal and transmitting section 67. Further, reference voltage source 65 is connected with switching circuit 60. This reference voltage source 65 may be a ground potential.

Controlling section 66 controls the entire operation of the blood test apparatus according to the present invention. The output of this controlling section 66 is connected with laser emitting apparatus 13, the controlling terminal of switching circuit 60, calculating section 63, transmitting section 67 and vacuuming means 14. Further, the input of controlling section 66 is connected with puncturing button 13j for performing puncturing by laser emitting apparatus 13, switch 13k that switches between laser puncturing and needle-puncturing, voltage detecting section 16a that detects the voltage of electric battery 16, skin detecting sensors 17d, timer 68 and detecting sensor 18y that detects attachment of needle unit 19. It may also be possible to connect and use a vacuum button that is manually pressed, instead of using skin detecting sensors 17j.

Next, the operation of electrical circuit section 15 will be explained. First, to which connectors 53a to 53f connection electrodes 41a to 45a and reference electrode 43c of sensor 22 and detecting sensor 18y are connected is detected. That is, according to a command from controlling section 66, a connector having zero electrical resistance with respect to the adjacent connectors is found among connectors 53a to 53f. Then, when the connector having zero electrical resistance is found, the connector is determined as connector 53 to be connected with reference electrode 43c. It is determined based on connector 53 connected with this reference electrode 43c that connectors 53 (i.e. starting with any of connectors 53a to 53f) are connected with connection electrodes 44a, 45a, 41a, 42a and 43a, respectively. In this way, connectors 53a to 53g respectively connected with connection electrodes 41a to 45a, reference electrode 43c and detecting sensor 18y are determined and then blood 8 is measured. Further, signals from detecting sensor 18y are connected to controlling section 66 through switching circuit 60.

In the measurement operation, switching circuit 60 is switched first to connect detection electrode 41 (see FIG. 11), which serves as an active electrode for measuring the amount of blood components, with current/voltage converter 61. Further, detection electrode 42, which serves as a sensing electrode for sensing the inflow of blood 8, is connected with reference voltage source 65. Then, a certain voltage is applied between detection electrode 41 and detection electrode 42. In this state, when blood 8 flows in, a current flows between detection electrode 41 and detection electrode 42. This current is converted into a voltage by current/voltage converter 61 and this voltage value is converted into a digital value in A/D converter 62. The digital value is outputted to calculating section 63. Calculating section 63 detects based on the digital value that sufficient blood 8 has flowed in. At this point, the operation of vacuuming means 14 is turned off.

Next, glucose, which is a blood component, is measured. To measure the amount of glucose components, according to a command from controlling section 66, switching circuit 60 is switched, and detection electrode 41, which serves as an active electrode for measuring the amount of blood components, is connected with current/voltage converter 61. Further, detection electrode 43, which serves as a counter electrode for measuring the amount of glucose components, is connected with reference voltage source 65.

While, for example, the glucose in blood and its oxidation-reduction enzyme are reacted for a certain period, current/voltage converter 61 and reference voltage source 65 are stopped. Further, after a certain reaction period (one to ten seconds) passes, a voltage (0.2 to 0.5 volts) is applied between detection electrodes 41 and 43 according to a command from controlling section 66. Then, a current flows between detection electrodes 41 and 43. This current is converted into the voltage in current/voltage converter 61, and the voltage value is converted into a digital value in A/D converter 62 and is outputted to calculating section 63. Calculating section 63 converts this digital value into an amount of glucose components.

Next, after the amount of glucose components is measured, the Hct (hematocrit) value is measured. The Hct value is measured as follows. First, switch circuit 60 is switched according to a command from controlling section 66. Then, detection electrode 45, which serves as an active electrode for measuring the Hct value, is connected with current/voltage converter 61. Further, detection electrode 41, which serves as the counter electrode for measuring the Hct value, is connected with reference voltage source 65.

Next, according to a command from controlling section 66, a certain voltage (2 to 3 volts) is applied between detection electrodes 45 and 41 from current/voltage converter 61 and reference voltage source 65. The current that is applied between detection electrodes 45 and 41 is converted into a voltage in current/voltage converter 61 and the voltage value is converted into a digital value in A/D converter 62. The digital value is outputted to calculating section 63. Calculating section 63 converts the digital value into an Hct value.

Using the Hct value and amount of glucose components acquired in this measurement, the amount of glucose components is corrected by the Hct value with reference to a calibration curve or calibration curve table determined in advance, and the correction result is displayed in display section 64. Further, the correction result may be transmitted from transmitting section 67 to the injection apparatus that injects insulin. Although a radio wave may be used for this transmission, transmission is preferably performed by optical communication that does not interfere with medical equipment.

By transmitting measurement data corrected in this way from transmitting section 67 to automatically set the dose of insulin to administer in the injection apparatus, the patient needs not to set the dose of insulin to administer, so that annoyance of setting the dose of insulin to administer is eliminated. Further, the dose of insulin can be set in the injection apparatus without artificial means, so that it is possible to prevent setting errors.

Although measurement of glucose is explained as an example, by replacing sensor 22, the present invention is also effective to measure other blood components such as the lactate acid level, and cholesterol, in addition to glucose.

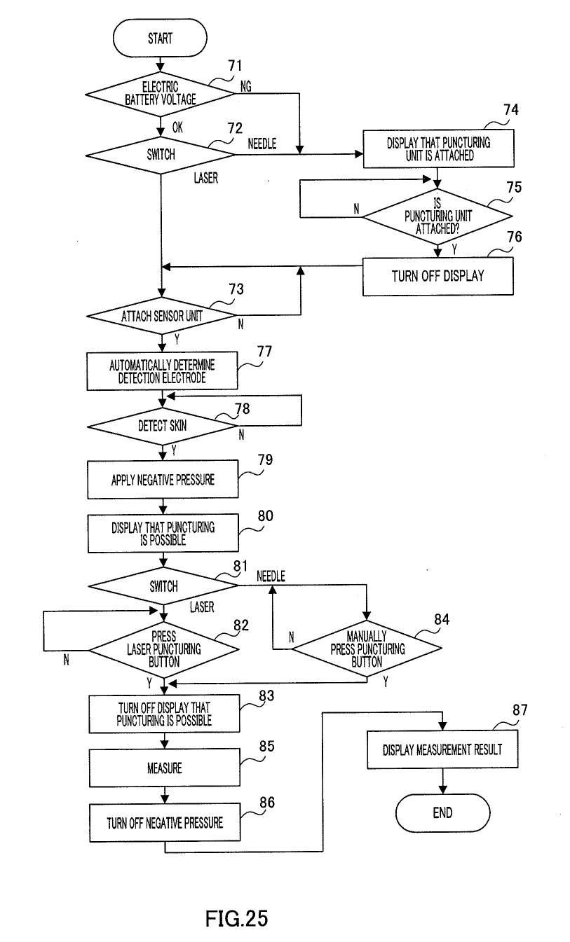

Next, the operation of blood test apparatus 11 will be explained using FIG. 25. When the power switch (not shown) is turned on, power is supplied to electrical circuit section 15. When power is supplied, the flow proceeds to step 71 and blood test apparatus 11 can detect the voltage of electric battery 16 in voltage detecting section 16a. This voltage detecting section 16a transmits the detection level and the result of detecting whether or not the voltage is a predetermined voltage level that allows laser puncturing.

When controlling section 66 decides that laser puncturing is possible, the flow proceeds to step 72. At this time, according to the detection level of voltage detecting section 16a, it is also possible to provide a plurality of selection modes in advance and automatically or manually switch between the laser emitting apparatus and the needle-puncturing apparatus according to the selected mode.

For example, as detection levels in the voltage detecting section, there are three selection modes based on a plurality of setting values of the remaining power of the electric battery and voltage determined in advance. Controlling section 66 selects between the following three modes based on the detection level in voltage detecting section 16a and the setting value of the selection mode set in advance. In the first selection mode, the detection level is equal to or more than the first setting value and the laser emitting apparatus is automatically selected. In the second selection mode, the detection level is equal to or more than a second setting value and less than the first setting value, and either the laser emitting apparatus or the needle-puncturing apparatus can be selected by the user. In the third selection mode, the detection level is less than the second setting value and the needle-puncturing apparatus is automatically selected. Further, the second selection mode (i.e. mode where the user can select either the laser emitting apparatus or the needle-puncturing apparatus) can be switched automatically or manually in advance. In case where either the laser emitting apparatus or the needle-puncturing apparatus is selected automatically, by making a setup in advance as to whether to select laser emitting apparatus 13 or needle-puncturing apparatus 14, the puncturing means is automatically switched without waiting for user to select when the mode switches to the second selection mode. At this time, when needle-puncturing apparatus 14 is selected as the puncturing means, use of laser emitting apparatus 13 built in the blood test apparatus is automatically prevented. That is, power supply to laser emitting apparatus 13 from electric battery 16 and control signals to laser emitting apparatus 13 are stopped or blocked by controlling section 66.

Then, in step 72, whether switch 13k is set to laser puncturing or needle-puncturing is detected. Further, as described above, in case where a setup is made such that puncturing means is selected automatically, the setup state is decided.

When the puncturing means is set to laser puncturing, the flow proceeds to step 73, and the blood test apparatus waits until sensor unit 17 is attached and shows a display that suggests attaching sensor unit 17. Further, attachment of this sensor unit 17 is detected when reference electrode 43c is detected. When sensor unit 17 is not attached, display section 64 shows a display that suggests attaching sensor unit 17. If puncturing unit 18 is attached and sensor unit 17 is not attached, sensor unit 17 is not electrically connected with reference electrode 43c, that is, sensor unit 17 is not electrically conducted with reference electrode 43c, so that electrical circuit section 15 built in the blood test apparatus can decide that sensor unit 17 is not attached. It naturally follows that the same applies when neither puncturing unit 18 nor sensor unit 17 is attached.

In a case where the voltage does not allow laser puncturing in step 71 and in a case where, even though the voltage allows laser puncturing, switch 13k is set to the needle-puncturing side in step 72, the flow proceeds to step 74 and display section 64 shows a display that suggests attaching puncturing unit 18, and then the flow proceeds to step 75. Further, whether or not puncturing unit 18 is attached is decided based on the output from detecting sensor 18y provided in puncturing unit 18. In this step 75, the blood test apparatus waits until puncturing unit 18 is attached. Here, when puncturing unit 18 is not attached after a predetermined time passes (this time is measured by timer 68), a warning means can make a warning.

When puncturing unit 18 is attached, the flow proceeds to step 76. Further, the display in step 74 that suggests attaching puncturing unit 18 is turned off, and the flow proceeds to step 73.

When attachment of sensor unit 17 is detected in step 73, the flow proceeds to step 77. In step 77, detection electrodes 41 to 45 are specified based on detected reference electrode 43c of sensor 22. Further, at the time reference electrode 43c is detected, the display in step 73 that suggests attaching sensor unit 17 is turned off.

Then, the blood test apparatus waits in step 78 until the blood test apparatus abuts on skin 7 to sample blood from. When skin detecting sensors 17d in sensor unit 17 detect skin 7, the flow proceeds to step 79 and vacuuming means 14 is operated. Then, this vacuuming means 14 applies a negative pressure to vacuuming chamber 14b (the vicinity of sensor 22). A vacuum button (not shown) may be connected with controlling section 66 and be pressed instead of using skin detecting sensors 23j.

When the current in the vacuum pump forming vacuuming means 14 changes or the time determined in advance in timer 68 passes, it is decided that skin 7 inside blood guiding part 34 is sufficiently lifted up, and the flow proceeds to step 80. In step 80, display section 64 displays that puncturing is possible. In step 81, when switch 13k selects the laser puncturing side, pressing of puncturing button 13j of laser emitting apparatus 13 is commanded in this display. Then, the flow proceeds to step 82 and laser emitting apparatus 13 waits until puncturing button 13j is pressed. When puncturing button 13j is pressed, the flow proceeds to step 83.

Further, when switch 13k selects the needle-puncturing side in step 81, pressing of puncturing button 18r of puncturing unit 18 is commanded in this display. Then, the flow proceeds to step 84 and puncturing unit 18 waits until puncturing button 18r is pressed. When puncturing button 18r is pressed, the flow proceeds to step 83.

In step 83, by pressing puncturing button 13j or puncturing button 18r, laser light 13h or puncturing needle 19a punctures skin 7. Blood 8 flows out as a result of puncturing skin 7. This blood 8 is taken in detecting section 37 of sensor 22. Then, in step 85, the blood sugar level of blood 8 is measured.

After the blood sugar level is measured in step 85, the flow proceeds to step 86 and the negative pressure from vacuuming means 14 is turned off. Then, the flow proceeds to step 87 and the blood sugar level that is measured is displayed in display section 64

Further, the display in step 80 to the effect that puncturing is possible, is turned off in step 83. That is, the display is turned off at the timing blood 8 reaches detection electrode 42 before the blood sugar level is measured in step 85. Further, the vacuuming may be turned off simultaneously at the timing blood 8 reaches detection electrode 42.

### Embodiment 2

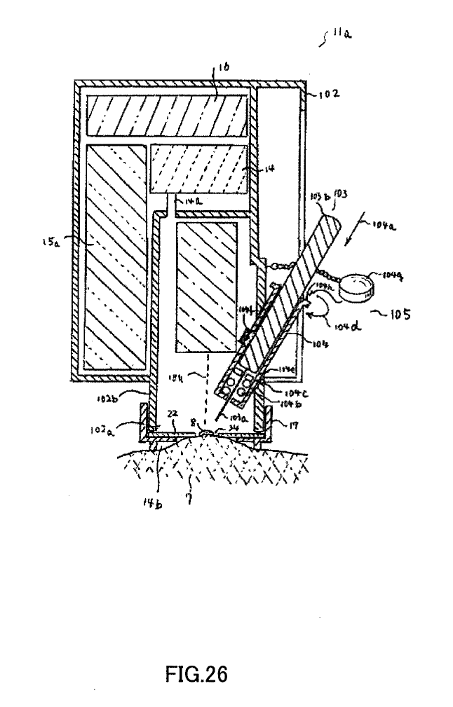

FIG. 26 is a cross-sectional view of blood test apparatus 11a according to Embodiment 2. While puncturing unit 18 is attached between puncturing opening part 12a and sensor unit 17 with Embodiment 1, puncturing needle part 103 corresponding to puncturing unit 18 is inserted from the oblique direction of housing 102 with Embodiment 2. Accordingly, Embodiment 2 will be explained mainly with this difference. Further, the same components as in Embodiment 1 will be assigned the same reference numerals and explanation thereof will be simplified.

In FIG. 26, housing 102 is made of a resin (corresponding to housing 12 of Embodiment 1), and one end of this housing 102 forms cylindrical body 102b of a cylindrical shape that has puncturing opening part 102a. Laser emitting apparatus 13 is attached inside this cylindrical body 102b. Further, vacuuming means 14a continuing to vacuuming passage 14a is attached to cylindrical body 102b. Further, electrical circuit section 15a (corresponding to electrical circuit section 15 of Embodiment 1) is provided next to cylindrical body 102b. Electric battery 16 is accommodated detachably at the end opposite to the end where puncturing opening part 102a is provided. Sensor unit 17 is attached detachably to puncturing opening part 102a.

Puncturing needle insertion part 104 is attached obliquely in the side surface of housing 102 and this puncturing needle insertion part 104 and puncturing needle part 103 constitute needle-puncturing apparatus 105. Puncturing needle part 103 is inserted in puncturing needle insertion part 104. Puncturing needle part 103 is inserted not to allow the negative pressure to escape from puncturing needle insertion part 104. A sealing member may be pasted for the same purpose.

Needle 103a attached to the front end of puncturing needle part 103 is provided to incline obliquely with respect to the optical axis of laser light 13h, and passes the center of blood guiding part 34 provided in the center of sensor 22 and punctures skin 7. That is, needle 103a punctures virtually the same position of skin 7 as the position punctured by laser light 13h.

By hitting puncturing needle part 103 in the direction of arrow 104a, needle 103a passes blood guiding part 34 and punctures skin 7. A little amount of blood 8 flows out from skin 7, this blood 8 is taken in sensor 22 and the property of this blood 8 is measured. 104b are springs that urge puncturing needle part 103 in the direction opposite to arrow 104a and functions to pull out puncturing needle 103a from skin 7.

In the surface (i.e. jointing surface) where cylindrical body 102b and puncturing needle insertion part 104 are attached, male screws and female screws are formed and, by rotating puncturing needle insertion part 103 in the direction of arrow 104d, it is possible to adjust the degree puncturing needle insertion part 104 intrudes into cylindrical body 102b. That is, by rotating puncturing needle insertion part 104 in the direction of arrow 104d or in the direction opposite to arrow 104d, it is possible to adjust the depth the needle punctures skin 7. Puncturing depth scales 104e are marked on the outer surface of puncturing needle insertion part 104.

Puncturing needle detecting sensor 104f is provided in puncturing needle insertion part 104 (corresponding to detecting sensor 18y of Embodiment 1), and the output of this puncturing needle detecting sensor 104f that detects insertion of puncturing needle part 103 in puncturing needle insertion part 104, is connected to electrical circuit section 15a (corresponding to electrical circuit section 15 of Embodiment 1).

Cap 104g is coupled to housing 102 with a chain and is provided attachably to rear end 104h of puncturing needle insertion part 104. In case where puncturing needle part 103 is not used, this cap 104g seals rear end 104h so as not to allow the negative pressure to escape.

As described above, a simple needle-puncturing apparatus is provided with the present embodiment, so that it is possible to make the puncturing unit small compared to puncturing unit 18 of Embodiment 1. Puncturing is possible by hitting rear end 103b of puncturing needle part 103 by the hand.

### Embodiment 3

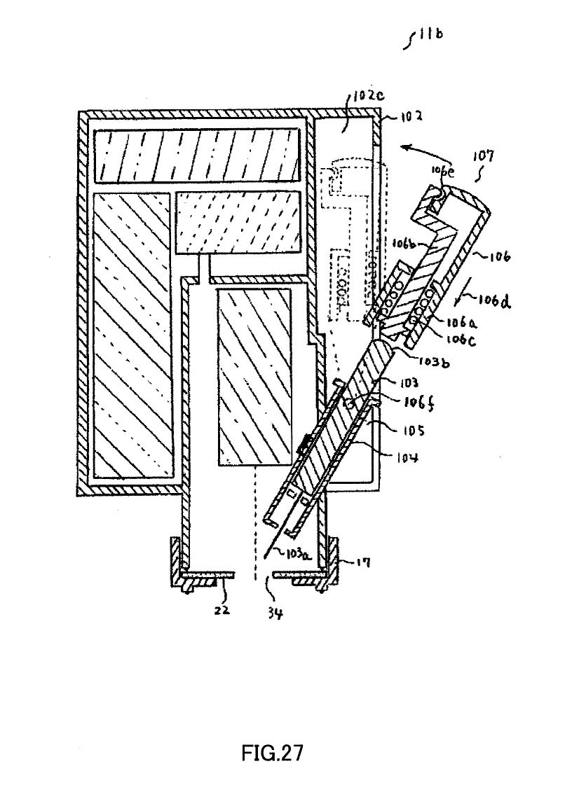

FIG. 27 is a cross-sectional view of blood test apparatus 11b according to Embodiment 3. While rear end 103b of puncturing needle part 103 constituting needle-puncturing apparatus 105 is hit by the hand with Embodiment 2, hammer unit 106 is attached to rear end 103b of puncturing needle part 103 to hit needle-puncturing apparatus 105 with Embodiment 3. Accordingly, Embodiment 3 will be explained mainly with this difference. Further, the same components as in Embodiment 2 will be assigned the same reference numerals and explanation thereof will be simplified.

In FIG. 27, 106a is a cylindrical body made of a resin and handle 106b is attached and slides back and forth inside this cylindrical body 106a. One end of this handle 106b is urged by springs 106c in the direction of arrow 106d. Further, the other end of handle 106b is held engagably by engaging part 106e.

By disengaging engaging part 106e, handle 106b is driven by springs 106c and is launched promptly in the direction of arrow 106d. Then, handle 106b hits rear end 103b of puncturing needle part 103. Then, needle 103a passes blood guiding part 34 of sensor 22 and punctures skin 7.

This cylindrical body 106a is attached to puncturing needle insertion part 104 to be rotatable about support point 106f. Consequently, when hammer unit 106 is not used, this hammer unit 106 can be accommodated in hollow part 102c (that is, the state shown by the dotted line) provided in the side surface of housing 102 as shown by the dotted line. Needle-puncturing apparatus 105 and hammer unit 106 constitute needle-puncturing apparatus 107. Further, when the laser puncturing apparatus switches to needle-puncturing apparatus 105 (including hammer unit 106), use of the laser puncturing apparatus is prevented and hammer unit 106 automatically rotates about support point 106f from hollow part 102c of the blood test apparatus and protrudes from the blood test apparatus. Then, hammer unit 106 is located in a position to drive needle-puncturing apparatus 105 (i.e. the state of FIG. 27).

As described above, the present embodiment differs from Embodiment 2 in having hammer unit 106, so that puncturing is possible by hitting puncturing needle part 103 under a certain condition. Further, when hammer unit 106 is not used, hammer unit 106 can be accommodated in hollow part 102c, which is convenient and not annoying when carrying the blood test apparatus.

### Embodiment 4

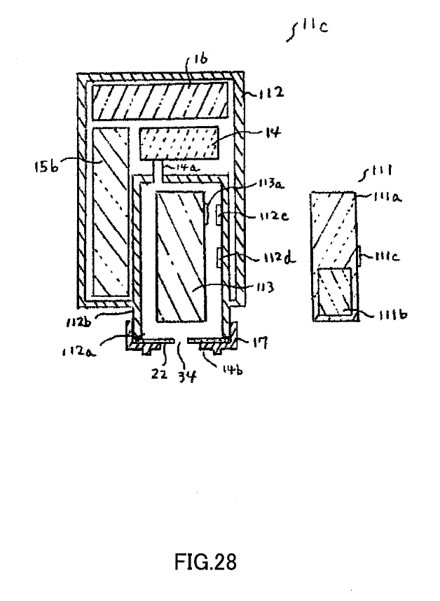

FIG. 28 is a cross-sectional view of blood test apparatus 11c according to Embodiment 4. While puncturing unit 18 is attached between puncturing opening part 12a and sensor unit 17 with Embodiment 1, puncturing unit 111, which is a needle-puncturing apparatus that performs puncturing by means of a puncturing needle, can be attached replacably in the location where laser emitting apparatus 113 (corresponding to laser emitting apparatus 13 of Embodiment 1) is attached with Embodiment 4. Accordingly, Embodiment 4 will be explained mainly with this difference. Further, the same components as in Embodiment 1 will be assigned the same reference numerals and explanation thereof will be simplified.

In FIG. 28, housing 112 is made of a resinic material (corresponding to housing 12 of Embodiment 1) and cylindrical body 112b of a cylindrical shape that has puncturing opening part 112a is provided with housing 112. Laser emitting apparatus 113 and any of puncturing unit 111 are attached replacably inside this cylindrical body 112b. Further, vacuuming means 14 continuing to vacuuming passage 14a is attached to cylindrical body 112b. Further, electrical circuit section 15b (corresponding to electrical circuit section 15 of Embodiment 1) is provided next to cylindrical body 112b. Electric battery 16 is accommodated replacably at the end opposite to the end where puncturing opening part 12a is provided. Sensor unit 17 is attached detachably to puncturing opening part 112a.

Puncturing unit 111 is constituted by hammer unit 111a and needle unit 111b attached detachably to this hammer unit 111a. Ferromagnetic member 111c identifies puncturing unit 111.

Ferromagnetic member 113a identifies laser emitting apparatus 113. Detecting sensor 112c that detects ferromagnetic member 113a adhered to laser emitting apparatus 113 and detecting sensor 112d that detects ferromagnetic body 111c adhered to puncturing unit 111 are attached inside cylindrical body 112b, and their outputs are connected to controlling section 66 inside electrical circuit section 15b. Consequently, electrical circuit section 15b can automatically identify whether either laser emitting apparatus 113 or puncturing unit 111 is inserted inside cylindrical body 112b.

In any case, laser light or a puncturing needle passes sensor unit 17 attached to puncturing opening part 112a and punctures skin 7, and, consequently same sensor 22 can be used. Further, puncturing is performed using a puncturing means that is built inside cylindrical body 112b, so that the blood test apparatus that is used becomes small compared to blood test apparatus 11 used in Embodiment 1.

Further, a vacuuming passage having the same function as vacuuming passage 18e explained in puncturing unit 18 of Embodiment 1 is formed in puncturing unit 111 and continues to the vacuuming means built in housing 112 of blood test apparatus 11c, so that it is possible to supply a negative pressure to vacuuming chamber 14b formed in sensor unit 17 without leaking the negative pressure. Further, a detecting sensor having the same function as detecting sensor 18y explained referring to puncturing unit 18 of Embodiment 1, is provided inside hammer unit 111a. Further, it is possible to carry puncturing unit 111 with blood test apparatus 11c by attaching puncturing unit 111 to the outer part of blood test apparatus 11c.

### Embodiment 5

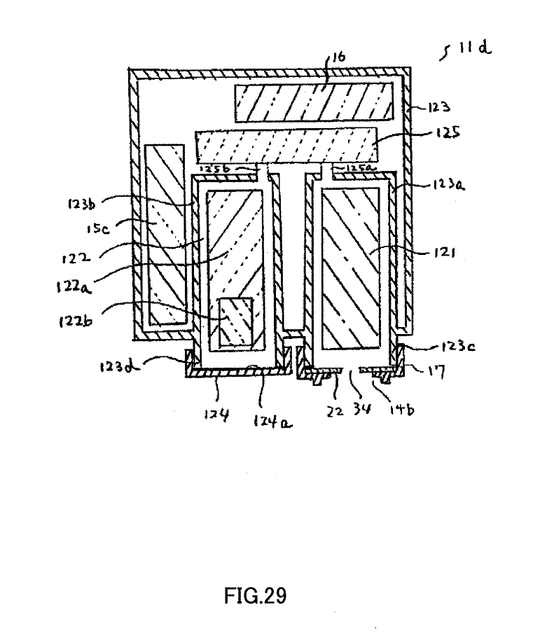

FIG. 29 is a cross-sectional view of blood test apparatus 11d according to Embodiment 5. While puncturing unit 18 is attached between puncturing opening part 12a and sensor unit 17 with Embodiment 1, both laser emitting apparatus 121 (corresponding to laser emitting apparatus 13 of Embodiment 1) and puncturing unit 122 (i.e. needle-puncturing apparatus) that performs puncturing by means of a puncturing needle, are attached inside same housing 123 with Embodiment 5. Accordingly, Embodiment 5 will be explained mainly with this difference. Further, the same components as in Embodiment 1 will be assigned the same reference numerals and explanation thereof will be simplified.

In FIG. 29, housing 123 is made of a resin (corresponding to housing 12 of Embodiment 1) and two cylindrical bodies 123a and 123b are provided side by side inside housing 123. Laser emitting apparatus 121 is built inside cylindrical body 123a and puncturing unit 122 is built inside cylindrical body 123b. Puncturing unit 122 is constituted by hammer unit 122a and needle unit 122b that is attached detachably to this hammer unit 122a.

Puncturing opening parts 123c and 123d of cylindrical bodies 123a and 123b are formed to allow sensor unit 17 to be detachably attached to puncturing opening parts 123c and 123d, and connectors 53a to 53f connected with electrical circuit section 15c are provided at the front end of puncturing opening parts 123c and 123d. Further, signals from the detecting sensor of the puncturing needle are connected directly to electrical circuit section 15c. Further, cap 124 can be attached to puncturing opening parts 123c and 123d. Electrically conductive plate 124a is pasted in the surface where this cap 124 abuts on puncturing opening part 123c or puncturing opening part 123d. Consequently, electrical circuit section 15c can decide whether sensor unit 17 is attached or cap 124 is attached, by detecting the electrically conducting states of adjacent connectors among connectors 53a to 53f.

Further, vacuuming means 125 (corresponding to vacuuming means 14 of Embodiment 1) continuing to vacuuming passages 125a and 125b is attached to cylindrical bodies 123a and 123b. Further, electrical circuit section 15c (corresponding to electrical circuit section 15 of Embodiment 1) is provided next to cylindrical body 123b. Electric battery 16 is accommodated replacably at the end opposite to the end where puncturing opening parts 123a and 123d are provided.

With blood test apparatus 11d according to the present embodiment, laser emitting apparatus 121 and puncturing unit 122 are attached in predetermined locations, so that the user does not leave laser emitting apparatus 121 and puncturing unit 122 when the user goes outside. Further, laser emitting apparatus 121 and puncturing unit 122 are built inside one housing 123 and, consequently, are convenient to carry. Furthermore, both puncturing means can use same sensor unit 17.

Further, a vacuuming passage having the same function as vacuuming passage 18e explained referring to puncturing unit 18 of Embodiment 1 is formed in puncturing unit 122, so that it is possible to supply a negative pressure to vacuuming chamber 14b formed in sensor unit 17 without leaking the negative pressure. Further, detecting sensor 122b having the same function as detecting sensor 18y explained referring to puncturing unit 18 of Embodiment 1, is provided inside hammer unit 122a.

The disclosure of Japanese Patent Application No. 2007-030017, filed on Feb. 9, 2007, including the specification, drawings and abstract, is incorporated herein by reference in its entirety.

### INDUSTRIAL APPLICABILITY

The present invention is applicable to a blood test apparatus that selectively uses a laser emitting apparatus that requires a supply of power and a needle-puncturing apparatus that does not require a supply of power as the puncturing means.

1. A blood test apparatus comprising:
a housing that comprises an open body of a cylindrical shape;
a blood sensor that can be attached detachably to the open open body of a cylindrical shape of the housing and that analyzes blood;
a blood guiding part that is formed in the blood sensor, that comprises an opening part and that stores blood which flows out from skin as a result of puncturing, in the opening part;
a laser emitting apparatus that is provided in the housing and that punctures skin by means of laser light which passes an interior of the open body of a cylindrical shape of the housing and the opening part of the blood guiding part and punctures skin; and
a needle-puncturing apparatus that is provided in the housing and that punctures skin by means of a needle which passes the opening part of the blood guiding part and punctures skin.

2. The blood test apparatus according to claim 1, wherein the needle-puncturing apparatus can be attached detachably to the open body of a cylindrical shape of the housing and comprises an open body of a same cylindrical shape as the housing.

3. The blood test apparatus according to claim 2, further comprising controlling means that, upon reception of a control signal for commanding execution of puncturing as input,
executes puncturing by means of the laser emitting apparatus, if the blood sensor is attached to the open body of a cylindrical shape of the housing, and
executes puncturing by means of the needle-puncturing apparatus, if the needle-puncturing apparatus is attached to the open body of a cylindrical shape of the housing.

4. The blood test apparatus according to claim 3, further comprising a voltage detecting section that detects a voltage of an electric battery that supplies power to the laser emitting apparatus,
wherein, when the voltage detected in the voltage detecting section is less than a predetermined setting level, the controlling means stops the execution of puncturing by means of the laser emitting apparatus.

5. The blood test apparatus according to claim 2, further comprising controlling means that detects which one of the blood sensor and the needle-puncturing apparatus is attached to the open body of a cylindrical shape of the housing, and displays a detection result.

6. The blood test apparatus according to claim 5, further comprising a voltage detecting section that detects a voltage of an electric battery that supplies power to the laser emitting apparatus,
wherein, when the voltage detected in the voltage detecting section is less than a predetermined setting level, the controlling means displays that puncturing is not possible by means of the laser emitting apparatus.

7. The blood test apparatus according to claim 1, further comprising:
a voltage detecting section that detects a voltage of an electric battery that supplies power to the laser emitting apparatus; and
controlling means that displays a selection mode for selecting one of the laser emitting apparatus and the needle-puncturing apparatus, according to a level of the voltage detected in the voltage detecting section.

8. The blood test apparatus according to claim 7, wherein the controlling section,
when the level of the voltage detected in the voltage detecting section is equal to or more than a predetermined setting level, displays that both the laser emitting apparatus and the needle-puncturing apparatus can be selected, and permits use of one apparatus alone selected by a user and prevents use of the other apparatus, and
when the level of the voltage detected in the voltage detecting section is less than the setting level, automatically selects the needle-puncturing apparatus, and permits use of the needle-puncturing apparatus and prevents use of the laser puncturing apparatus.

9. The blood test apparatus according to claim 7, wherein the controlling means,
when the level of the voltage detected in the voltage detecting section is equal to or more than a first setting level, automatically selects the laser emitting apparatus and permits the use of the laser emitting apparatus alone,
when the level of the voltage detected in the voltage detecting section is equal to or more than a second setting level and less than the first setting level, displays that both the laser emitting apparatus and the needle-puncturing apparatus can be selected, and permits use of one apparatus alone selected by a user and prevents use of the other apparatus, and
when the level of the voltage detected in the voltage detecting section is less than the second setting level, automatically selects the needle-puncturing apparatus, and permits use of the needle-puncturing apparatus alone and prevents use of the laser puncturing apparatus.

10. The blood test apparatus according to claim 1, further comprising:
a first puncturing unit that can be attached detachably between a sensor unit to which the blood sensor is attached and the open body of a cylindrical shape of the housing, and that forms a needle-puncturing apparatus; and
vacuuming means that applies a negative pressure to an internal space formed by the open body of a cylindrical shape of the housing and skin which is abutted by a front end of the open body of a cylindrical shape of the housing,
wherein an upper surface and a lower surface of the first puncturing unit are sealed, and a vacuuming passage communicates the upper surface with the lower surface of the first puncturing unit.

11. The blood test apparatus according to claim 10, wherein:
connector electrodes are formed at equal intervals in the upper surface and the lower surface of the first puncturing unit, wherein:
a first guide that fits in a puncturing opening part is formed in an upper part of the first puncturing unit; and a second guide that fits in the sensor unit is formed in a lower part of the first puncturing unit.

12. The blood test apparatus according to claim 1, wherein a needle unit is provided removably in the first puncturing unit.

13. The blood test apparatus according to claim 1, wherein the needle-puncturing apparatus is provided to incline obliquely with respect to an optical axis of laser light emitted from the laser emitting apparatus.

14. The blood test apparatus according to claim 13, wherein a second puncturing unit, one end of which a puncturing needle is attached to, is inserted removably in the needle-puncturing apparatus.

15. The blood test apparatus according to claim 14, wherein a hammer unit that hits the other end of the second puncturing unit is attached.

16. The blood test apparatus according to claim 15, wherein the hammer unit is provided so as to be accommodated inside the housing.

17. The blood test apparatus according to claim 1, wherein one of the laser emitting apparatus and the needle-puncturing apparatus is attached inside the housing.

18. The blood test apparatus according to claim 17, further comprising:
detecting means that detects that one of the laser emitting apparatus and the needle-puncturing apparatus is attached inside the housing; and
identifying means that identifies which one of the laser emitting apparatus and the needle-puncturing apparatus is attached.

19. The blood test apparatus according to claim 1, wherein:
both the laser emitting apparatus and the needle-puncturing apparatus are attached inside the housing; and
use of one of the laser emitting apparatus and the needle-puncturing apparatus is permitted.

20. The blood test apparatus according to claim 19, further comprising detecting means that detects to which one of the laser emitting apparatus and the needle-puncturing apparatus the blood sensor is attached.
